(19) United States
(12) Patent Application Publication
(10) Pub. No.: US 2004/0052680 A1
(43) Pub. Date: Mar. 18, 2004

(54) Treatment of environmental unit atmosphere and/or interior with generation of radicals

(76) Inventors: Bryan M. Elwood, Candler, NC (US); Walter Jeff Tipton, Asheville, NC (US)

Correspondence Address: BAKER + HOSTETLER LLP, WASHINGTON SQUARE, SUITE 1100, 1050 CONNECTICUT AVE. N.W., WASHINGTON, DC, 20036-5304, US

(21) Appl. No.: 10/245,307

(22) Filed: Sep. 18, 2002

(51) Int. Cl. A61L 2/00; A01N 1/00

(52) U.S. Cl. 422/24; 422/121

(57) ABSTRACT

An apparatus is provided for decontaminating, sterilizing, or disinfecting an environmental unit chamber and its atmosphere that includes a filter, an air pump, a radical generator, an environmental unit, and an airflow ductwork system. The radical generator is capable of generating radicals selected from the group consisting of singlet oxygen ions, hybrid ozone, ionized oxygen, ionized nitrogen, hydroxyls, and hydrogen peroxide. A method for decontaminating, sterilizing, or disinfecting an environmental unit chamber and its atmosphere using radicals are provided.

### FIELD OF THE INVENTION

The present invention relates generally to methods and apparatuses for treating the atmosphere and/or interior of an environmental unit such as, for example, an incubator. More particularly, the present invention relates to methods and apparatuses to decontaminate, sterilize, and/or disinfect the atmosphere in the unit as well as the interior surfaces of the chamber of the unit itself.

### BACKGROUND OF THE INVENTION

Environmental units are widely used in industrial and laboratory applications, including, for example, incubators that are used to ensure the safety and/or performance of a cell culture environment for research, clinical and/or life science applications.

In the use of environmental units, it is often necessary to decontaminate, sterilize, and/or disinfect the interior of the unit and the atmosphere inside the unit before introducing the materials to be stored in the unit. One way in which this treatment is achieved is by heating the interior of the unit to a temperature sufficient to decontaminate the unit and its atmosphere. In another treatment method, the interior of the unit is manually scrubbed with chemicals that decontaminate, sterilize, and/or disinfect the surfaces of the unit.

These known decontamination methods have some drawbacks. For example, treatment by heat often requires substantial periods of time, sometimes up to 24 hours. A high temperature may be required, and the materials in the unit generally must be tolerant of the high heat levels and the wide temperature cycles. Also, a time period for temperature stabilization from the hot treatment temperature to the lower operating temperature is required.

Treatment by scrubbing with chemicals suffers from the disadvantage that it sometimes may not decontaminate, sterilize, or disinfect all surfaces of the unit completely, particularly, in the case of rough surfaces or microscopic cracks. Also, the need to handle chemicals may be undesirable for some.

Accordingly, an apparatus and method that decontaminates, sterilizes, or disinfects the unit and/or its atmosphere in less time than is currently required by heat decontamination is desired. In addition, an apparatus and method that decontaminates, sterilizes, or disinfects the unit and/or its atmosphere more thoroughly than the traditional method of chemical scrubbing is also desired.

### SUMMARY OF THE INVENTION

It is therefore a feature and advantage of the present invention to provide an apparatus comprising a radical generator and an airflow ductwork system to introduce radicals into an environmental unit having an internal chamber.

It is another feature and advantage of the present invention to provide a method of decontaminating, sterilizing, and/or disinfecting an environmental unit and/or its atmosphere by generating radicals and introducing the radicals into a chamber of the environmental unit.

It is another feature and advantage of the present invention to provide an apparatus for decontaminating, sterilizing and/or disinfecting an environmental unit and/or its atmosphere having means for generating radicals and means for introducing the radicals into a chamber of the environmental unit.

The above and other features and advantages are achieved through the use of a novel decontaminating apparatus and method as herein disclosed. In accordance with one embodiment of the present invention, an apparatus for decontaminating, sterilizing, and/or disinfecting an environmental unit is provided. The apparatus includes a ductwork system and a radical generator. Additionally, the ductwork system may comprise a filter and/or an air pump so that the filter, air pump, and radical generator are connected such that the radicals that are generated can be introduced into the environmental unit airflow ductwork system and environmental unit chamber. The ductwork system may also comprise an electrostatic generator and/or an air compressor. The environmental unit may also comprise a fan and/or additional ductwork. The radical generator is capable of generating radicals selected from the group consisting of singlet oxygen ions, hybrid ozone, ionized oxygen, ionized nitrogen, hydroxyls, and hydrogen peroxide.

There has thus been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a schematic diagram illustrating an apparatus for treating an environmental unit according to a first preferred embodiment of the present invention.

FIG. 2 is a flow chart illustrating a method for treating an environmental unit by generating radicals and, introducing the radicals into the atmosphere of an environmental unit chamber, according to a preferred embodiment of the present invention.

FIG. 3 is a schematic diagram of an apparatus for treating an environmental unit according to a second preferred embodiment of the present invention.

### DETAILED DESCRIPTION OF PREFERRED EMBODIMENTS OF THE INVENTION

The present invention provides apparatuses and methods suitable for generating radicals from atmospheric air and directing the radicals into the chamber of an environmental unit, such as, for example, an incubator. Examples of the environmental units can include incubators that may be capable of maintaining steady and uniform levels of carbon dioxide (CO₂), temperature, or relative humidity inside the chamber of the environmental unit. That is, the CO₂ concentration, temperature, or relative humidity may be controlled and maintained at a more or less constant level that is more or less uniform throughout the chamber of the environmental unit. The environmental unit may have a water jacket surrounding the chamber to assist in maintaining thermal stability and uniform heating.

In some embodiments, decontaminating, sterilizing, and/or disinfecting the chamber of the unit and/or the air inside is accomplished by delivering radicals through a ductwork system into the interior chamber of the environmental unit. The radical generator has an ultraviolet light bulb, a stainless steel pipe, and a magnetic core.

The radical generator comprises an ultraviolet light bulb, a stainless steel pipe, and a magnetic core. The ultraviolet light bulb emits light with a wavelength of approximately 184 nm. This wavelength of light is capable of generating singlet oxygen ions, hybrid ozone, ionized oxygen, ionized nitrogen, hydroxyls, and hydrogen peroxide from the oxygen (O₂), nitrogen (N₂), and water vapor (H₂O) found in atmospheric air. One such light bulb is disclosed in U.S. Pat. No. 4,700,101 and is used in radical generators such as the commercially available NIOX™ generator from LaundrOx, Inc.

The ductwork system preferably has a filter and an air pump. The ductwork system may also comprise an electrostatic generator and/or an air compressor to pressurize the air in the ductwork system and/or the chamber to effect more rapid treatment of the chamber and the air. The environmental unit may also comprise a fan and/or additional ductwork to enhance performance by improving the circulation of the radically charged air.

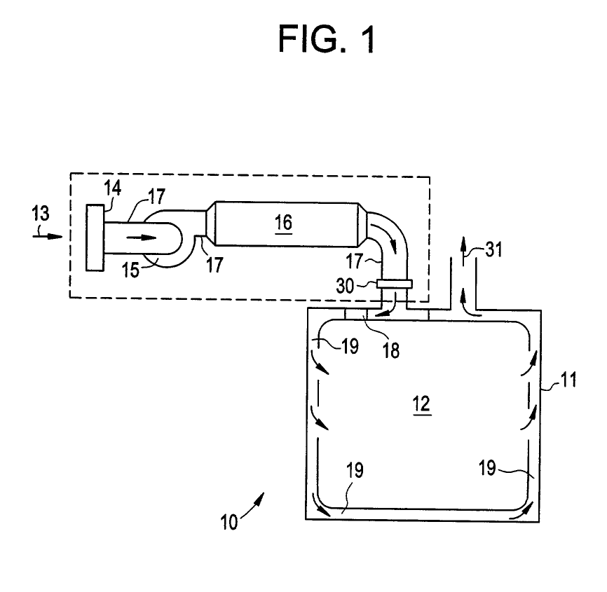

A first preferred embodiment of the present inventive apparatus and method is illustrated in FIG. 1. The apparatus 10 comprises a treatment module 13 for use with an environmental unit 11. A treatment module 13 comprises a filter 14, an air pump 15, and a radical generator 16. It is understood that the components of the module 13 may be placed in any order within the module 13 and be within the scope and spirit of the present invention. FIG. 1 is intended to illustrate one such ordering of the filter 14, air pump 15, and radical generator 16 within the module 13.

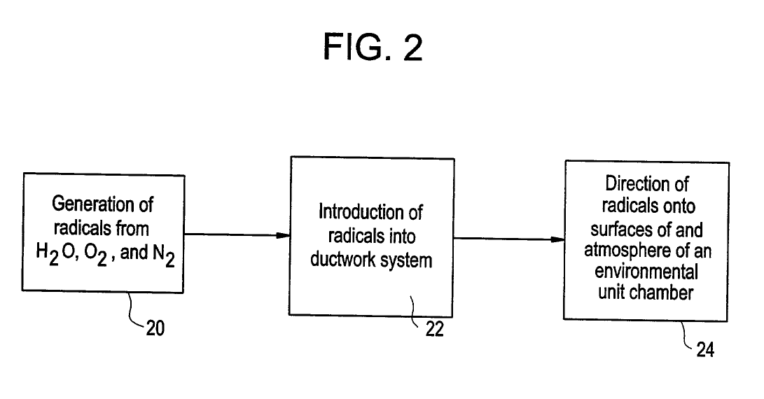

As shown in FIG. 1, the components of the module 13 are connected by a ductwork system 17 that forms an open loop treatment system As illustrated in FIG. 1, atmospheric air containing oxygen (O₂), nitrogen (N₂), and water vapor (H₂O) enters the filter 14, which serves to filter solid contaminants from the air. The air is directed through the ductwork system 17 via the air pump 15. The air then enters the radical generator 16 where it is exposed to the light generated by an ultraviolet light bulb, preferably at approximately 184 run. This step is illustrated at step 20 in FIG. 2.

The air, as it exits the radical generator 16 and continues through the ductwork 17, is charged with radicals selected from the group consisting of singlet oxygen ions, hybrid ozone, ionized oxygen, ionized nitrogen, hydroxyls, and hydrogen peroxide. This step is illustrated at step 22 in FIG. 2.

This radically charged air is directed into the chamber 12 of the environmental unit 11 in order to treat the chamber 12 and the atmosphere of the environmental unit 11. This step is illustrated at step 24 in FIG. 2. This treatment serves to decontaminate, sterilize, and/or disinfect the chamber 12 and/or the atmosphere of the unit 11.

In yet another preferred embodiment, the environmental unit 11 comprises a fan 18 to enhance performance by improving the circulation of the radically charged air. In still another more preferred embodiment, the environmental unit 11 comprises additional ductwork 19 to enhance performance by improving the circulation of the radically charged air.

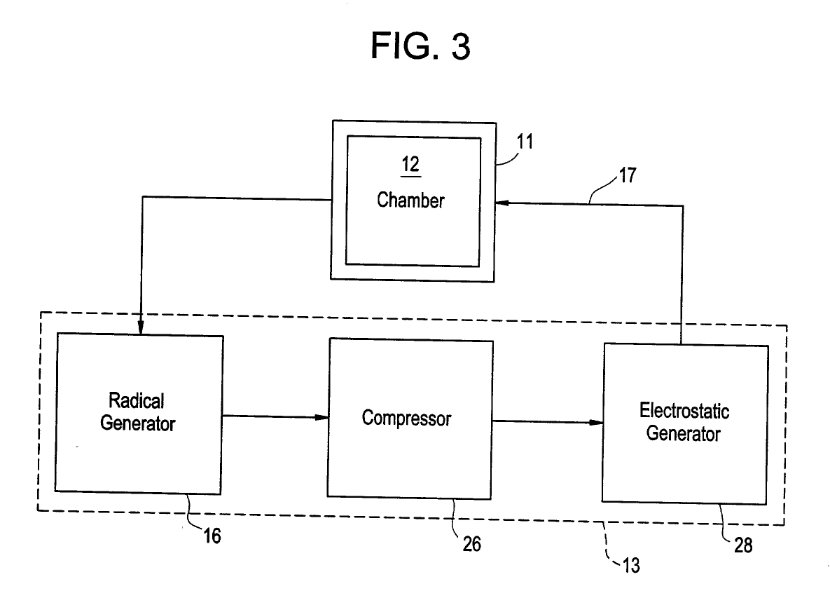

As illustrated in FIG. 3, in a more preferred embodiment, the ductwork 17 comprises an air compressor 26 to pressurize the air in the ductwork system 17 and/or the chamber 12 to effect more rapid treatment of the chamber and the air. As also illustrated in FIG. 3, in another more preferred embodiment, the ductwork 17 comprises an electrostatic generator 28 to increase the effectiveness of the radicals in treating the chamber 12 and the atmosphere of the environmental unit 11.

It is understood that the airflow pattern may also form a closed loop and be within the scope and spirit of the present invention. FIG. 3 illustrates a second preferred embodiment having an environmental unit 11, a radical generator 16, a compressor 26, and an electrostatic generator 28. The compressor 26 and electrostatic generator 28 can be implemented in both open loop and closed loop embodiments.

It will be appreciated that the present invention can be implemented utilizing the radical generator in a treatment module 13 that can be selectively connected to an environmental unit 11 for the treatment process. For example, the treatment module 13 can be a portable unit that can include the radical generator 16, air pump 15, and filter 14 shown in FIG. 1. A coupling arrangement 30 can be provided to removably couple the module 13 to the pre-existing ductwork 19 already present in the environmental unit.

In the embodiment illustrated in FIG. 1, the treatment module 13 comprising the radical generator 16, air pump 15, and filter 14 is coupled to the environmental unit 11 when treatment is desired and directs radical treated air into the existing duct work system 17. If no existing ductwork 19 is present, the module 13 can simply direct air into the chamber 12 via a port at the coupling 30. An exit vent 31 is provided to permit the air in the chamber 12 to exit. In this open loop arrangement, the treatment system 13 is run for a sufficient length of time for untreated air to be exhausted and replaced with treated air. After the treatment cycle is complete, the treatment module 13 can be disconnected and removed. In situations where the existing ductwork 19, (closed or open loop) is already present for temperature maintenance and other treatment of the air inside the chamber, the treatment module 13 can be uncoupled from the circulation system and removed as desired after treatment. The module 13 can similarly be uncoupled and removed where no existing ductwork 19 is present.

FIG. 3 shows a second embodiment in which a treatment module 13 tool includes the radical generator 16, an air compressor 26 and an electrostatic generator 28. A duct 17 leading from the electrostatic generator 28 directs air into the chamber 12. Air exiting from the chamber 12 is directed into the radical generator 16 as shown. It is understood that the elements of the treatment module 13 may be arranged and connected in any order suitable for directing radically charged air through the ductwork 17 and into the chamber 12. This embodiment can also be implemented with the treatment module 13 as a portable treatment tool that comprises the radical generator 16, compressor 26 and electrostatic generator 28. A benefit of embodiments in which a portable treatment tool is utilized is that the cost of the radical generator is located in one portable treatment tool. This treatment tool can be utilized to treat large numbers of environmental units, one at a time. Therefore, a facility having multiple environmental units can be served with a single treatment device if desired. Further, the treatment device can be moved from facility to facility, so that a large number of environmental units located in various facilities can be treated one at a time with a single treatment device.

Although the preferred embodiments are described in the context of a detachable, removable, portable treatment module 13 that is connected in either an open loop or closed loop mode to existing ductwork 19 in an environmental unit 11 such as an incubator, the treatment device can alternatively be partially or fully integrated with the environmental unit 11. In such embodiments, the components of the treatment module 13 can be provided in more or less permanent connection with the other components of the environmental unit such as, for example, the existing air circulation system.

In other embodiments any or all of the other elements of the module (such as e.g., the air pump, compressor, filter or ductwork) can be emitted. For example, in one embodiment the radical generator 16 can be located inside the existing structure of the environmental unit 11, by itself, or with the additional components.

The many features and advantages of the invention are apparent from the detailed specification, and thus, it is intended by the appended claims to cover all such features and advantages of the invention which fall within the true spirits and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. An apparatus for use with an environmental unit having a chamber, comprising:
a ductwork system that provides air to the chamber wherein the radical generator is provided in the ductwork system;
a radical generator that generates radicals with ultraviolet radiation and directs them into the chamber.

2. The apparatus of claim 1, further comprising a ductwork system that provides air to the chamber wherein the radical generator is provided in the ductwork system.

3. The apparatus of claim 1, wherein the radical generator generates radicals selected from the group consisting of singlet oxygen ions, hybrid ozone, ionized oxygen, ionized nitrogen, hydroxyls, and hydrogen peroxide.

4. The apparatus of claim 1, wherein the apparatus directs air to fill the volume of the environmental unit chamber.

5. The apparatus of claim 1, wherein the apparatus directs air onto the surfaces of the environmental unit chamber.

6. The apparatus of claim 1, wherein the radical generator decontaminates, sterilizes, and/or disinfects the air before it enters the environmental unit chamber.

7. The apparatus of claim 1, wherein the apparatus additionally comprises an air pump.

8. The apparatus of claim 1, wherein the apparatus additionally comprises a fan.

9. The apparatus of claim 1, wherein apparatus additionally comprises a filter.

10. The apparatus of claim 1, wherein the apparatus additionally comprises an air compressor.

11. The apparatus of claim 1, wherein the apparatus additionally comprises an electrostatic generator.

12. The apparatus of claim 1, wherein the apparatus additionally comprises ductwork in the environmental unit.

13. The apparatus of claim 1, where the radical generator is external to the environmental unit.

14. The apparatus of claim 1, wherein the radical generator is external to the environmental unit.

15. An apparatus for use with an environmental unit having a chamber, comprising:
means for generating radicals with ultraviolet radiation; and
means for directing the radicals into the chamber.

16. The apparatus of claim 15, wherein the radical generating means generates radicals selected from the group consisting of singlet oxygen ions, hybrid ozone, ionized oxygen, ionized nitrogen, hydroxyls, and hydrogen peroxide.

17. The apparatus of claim 15, wherein the means for directing directs air having the radicals to fill the volume of the environmental unit chamber.

18. The apparatus of claim 15, wherein the means for directing directs air having the radicals onto the surfaces of the environmental unit chamber.

19. The apparatus of claim 15, wherein the radical generator decontaminates, sterilizes, and/or disinfects the air before it enters the environmental unit chamber.

20. The apparatus of claim 15, wherein the apparatus additionally comprises an air pump.

21. The apparatus of claim 15, wherein the apparatus additionally comprises a fan.

22. The apparatus of claim 15, wherein the apparatus unit additionally comprises a filter.

23. The apparatus of claim 15, wherein the apparatus additionally comprises an air compressor.

24. The apparatus of claim 15, wherein the apparatus additionally comprises an electrostatic generator.

25. The apparatus of claim 15, wherein the apparatus additionally comprises ductwork in the environmental unit.

26. A method for treating an environmental unit chamber comprising:
(a) generating radicals with ultraviolet radiation; and
(b) directing the radicals into the environmental unit chamber.

27. The method of claim 26, wherein the radicals generated are selected from the group consisting of singlet oxygen ions, hybrid ozone, ionized oxygen, ionized nitrogen, hydroxyls, and hydrogen peroxide.
